(19) United States
(12) Patent Application Publication
(10) Pub. No.: US 2024/0199300 A1
(43) Pub. Date: Jun. 20, 2024

(54) INDICATOR FOR PERISHABLE PRODUCTS

(71) Applicant: CMeye AS, Olso (NO)

(72) Inventors: Davoud TAYEBI, Oslo (NO); Lars KILAAS, Trondheim (NO)

(21) Appl. No.: 18/286,902

(22) PCT Filed: Apr. 8, 2022
(86) PCT No.: PCT/NO2022/050083

§ 371 Date: Oct. 13, 2023

(30) Foreign Application Priority Data
Apr. 14, 2021 (NO) 20210465

(51) Int. Cl. B65D 79/02; G01K 3/04; G01K 11/16; G01N 21/78; G01N 31/22; G01N 33/04; G01N 33/08; G01N 33/14; G01N 33/15; G01N 33/52

(52) U.S. Cl. CPC B65D 79/02; G01K 3/04; G01K 11/16; G01N 21/78; G01N 31/229; G01N 33/04; G01N 33/08; G01N 33/14; G01N 33/15; G01N 33/52

(57) ABSTRACT

The present disclosure concerns an indicator (1) for a perishable product (8), the indicator (1) comprising indicator means, configured to indicate an exposure to an undesired condition. The indicator (1) further comprises a shell (1a) encapsulating the indicator means. The indicator (1) is configured to be immersed and freely-moveable in the perishable product (8).

### TECHNICAL FIELD

The present invention is concerned with an indicator for perishable products, the use of an indicator for perishable products and a method for production of an indicator for perishable products.

### BACKGROUND

Perishable products comprise fluid or granular products that may spoil when exposed to an undesirable condition. Such an undesirable condition may, for instance be a temperature outside an acceptable range, such as a too high temperature or a too low temperature. Other undesirable conditions are, for instance, exposure to UV light or to IR light, a too high pressure or too low pressure, bacterial or fungal contamination or exposure to radioactivity. Perishable products are utilized across a range of industries and may comprise granular materials, such as powders or particles, or fluids, such as liquids, emulsions or colloidal dispersions. In the medical field, perishable products may comprise vaccines, medicines, e.g. insulin, blood, intravenous fluids or biological samples, such as blood samples or saliva samples. In the field of food technology perishable products may comprise dairy products, egg-based fluids or beverages, such as wines or fresh juices. In the field of chemicals perishable products may comprise various compositions. As an example, temperature control and handling of chemical initiators used in radical polymerization reactions is crucial for avoiding fire or explosions. In most cases, particular transport and storage technology is used, to safely transport the perishable product from the production location to the end-user. For example, vaccines are temperature sensitive and are handled with cold chain technology. Thereby, the vaccine is kept within an acceptable temperature range during transport and storage, to avoid spoiling.

Nevertheless, the perishable product may unintentionally be exposed to an undesired condition. For instance, cooling installations may malfunction, insufficient sterilization may occur, or human errors may arise during handling of the perishable product. Often, it is not apparent from visual observation of the perishable product that spoiling has occurred. Especially for perishable products in the medical field, such as vaccines or medicines, failing to detect spoiling may have severe or even life-threatening consequences. Therefore, it is essential to monitor and signal whether the perishable product was exposed to an undesired condition during storage and transport.

One way of monitoring conditions during storage and transport is to include an indicator in the bulk package holding a batch of containers with a perishable product. The bulk package may, for instance, be a cold storage box and the indicator a temperature indicator. Based on a visual indication by the temperature indicator, an operator can quickly determine whether the batch has been spoiled and should be discarded. In this case, however, individual containers are not monitored and therefore an end-user has no information concerning the individual container at hand.

Another solution for monitoring conditions during storage and transport is therefore to include an indicator in or on each individual container holding the perishable product. Known indicators may be attached to the outside or to the inside of the container. Indicators placed on the inside may, for instance, be attached to the lid or to the bottom of the container. The indicator is then adhered to the lid or the bottom wall of the container during the container production process and before the filling of the container with a perishable product. Disadvantageously, the production process thereby becomes more complicated and time-consuming and existing production lines must be adapted to allow for this additional production step. Alternatively, the indicator may be moulded as an integral part of the container. However, this requires moulding of a complex geometry of the container or container lid, requiring complex and error-prone moulding steps.

Alternative solutions for monitoring conditions of products are provided in the following patent applications. US2009/0266290 A1 relates to a sensing device for detecting the presence or absence of chemical species in beverages or food items. The device comprises a sensing material. The indicator is for placement in the beverages or food items by the user and floats at the surface of the product. There is no protection of the sensing material. EP0932040 is related to the use of indicator dyes that change color during pH changes, to monitor the freshness of food and beverages. Indicator dyes from the group of anthocyanins are suggested and are included in an indicator foil coated on a polymer. The indicator dye is in direct contact with the product to be monitored. US2008/296191 is directed towards a complex packaging comprising one or more integrated sensors, such as a first sensor arranged in the main body of the package. The package further comprises a display unit and a processing unit.

Consequently, there exists a clear need for a solution wherein conditions during storage and transport can reliably be monitored in each individual container with a perishable product, without the disadvantages of a complicated, error-prone and/or time-consuming container production process.

### SUMMARY

The present disclosure concerns an indicator for a perishable product according to claim 1. The present invention also concerns the use of an indicator and a method for producing an indicator, according to the claims and as further disclosed herein.

### FIGURES

FIG. 1A schematically shows an indicator according to the disclosure.

FIG. 1B schematically shows an alternative indicator according to the disclosure.

FIG. 1C schematically shows a second alternative indicator according to the disclosure.

FIG. 1D schematically shows a third alternative indicator according to the disclosure.

FIG. 1E schematically shows a fourth alternative indicator according to the disclosure.

FIG. 2A schematically shows a longitudinal cross-section of a container according to the disclosure.

FIG. 2B schematically shows a transversal cross-section of a container according to the disclosure.

FIG. 3A shows a vaccine container with an indicator according to the disclosure that has not changed color.

FIG. 3B schematically shows a vaccine container with an indicator according to the disclosure that has changed color.

FIG. 4A shows a container in the form of a syringe with a ring shaped indicator according to the disclosure that has not changed color.

FIG. 4B shows a container in the form of a syringe with a ring-shaped indicator according to the disclosure that has changed color.

FIG. 5A shows a container in the form of a syringe with cylinder shaped indicator according to the disclosure that has not changed color.

FIG. 5B shows a container in the form of a syringe with cylinder shaped indicator according to the disclosure that has changed color.

### DETAILED DESCRIPTION

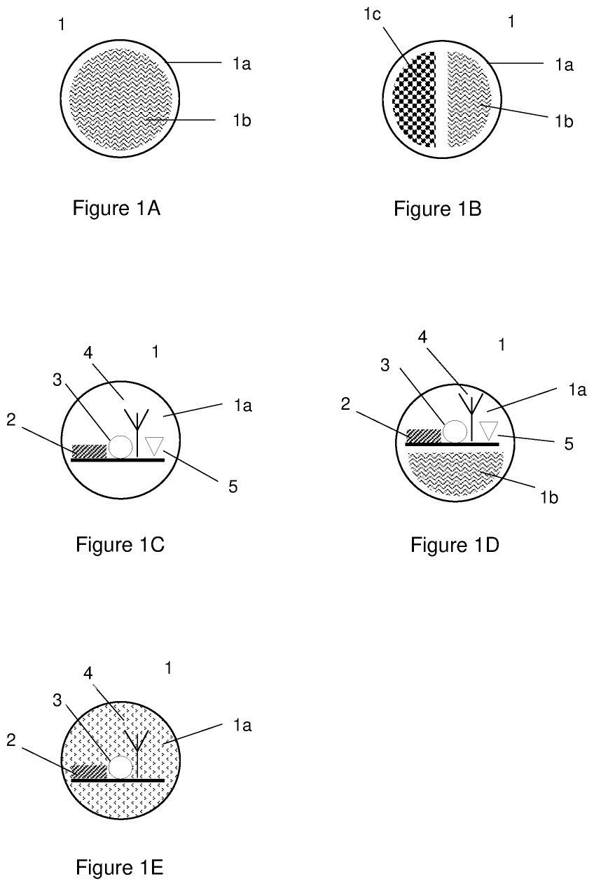

An indicator 1 according to the disclosure is schematically shown in FIG. 1A. The indicator 1 comprises indicator means, configured to indicate an exposure to an undesired condition; and a shell 1a encapsulating the indicator means. The indicator 1 is configured to be immersed and freely-moveable in a perishable product 8. When exposed to the undesired condition the perishable product 8 may become spoiled. The undesired condition may be a temperature outside an acceptable temperature range, a pressure outside an acceptable pressure range, light with an undesirable wavelength, such as UV light or IR light, radioactivity and/or bacterial-, fungal- or particulate-contamination. Preferably the undesired condition is either temperature or pressure outside an acceptable range, or light with an undesirable wavelength, and most preferably the undesired condition is temperature outside an acceptable temperature or temperature setpoints, or desired temperature ranges, or desired temperature intervals.

The shell 1a comprises the outer surface of the indicator 1. The shell 1a forms a barrier between the indicator substance(s), further defined below, of the indicator means and the perishable product. The shell 1a is preferably fluid-tight, or hermetically tight. In one embodiment, the seal forms a hermetically sealed encapsulation of the indicator means. In a preferred embodiment, there is no contact, either physically or chemically, between the indicator means and the product. The shell 1a may be non-transparent, piecewise-transparent, transparent or translucent. Preferably the shell is piecewise transparent, transparent or translucent. By piecewise transparent is meant that at least a section of the shell, such as forming one or more windows in the shell, is transparent. By the term transparent is meant that all, or nearly all, light will pass through it. By the term translucent means that some, but not all, light passes through the shell. In one embodiment, the indicator comprises a shell (1a) which is fluid-tight or hermetically sealed, and either transparent, piecewise transparent, or translucent, encapsulating the indicator means. Preferably, the indicator comprises a fluid-tight and transparent, piecewise transparent, or translucent shell (1a) encapsulating the indicator means. The shell 1a preferably comprises an inorganic polymer, g. glass, metal oxides, an organic polymer or a combination thereof. The shell 1a may comprise an organic polymer, such as selected from the group of acrylonitrile, butadiene styrene, polyamide, polycarbonate, polyester, vinyl esters, epoxy systems, polyether, polyethylene, polylactic acid, polypropylene, polyvinyl chloride, silicone and urethane, or a combination thereof. The described polymers are meant as examples for suitable polymers and hence the invention is not limited for use of only these systems. The indicator 1 may have a spherical shape, a cuboid shape, a cylindrical shape, a pyramidal shape, a conical shape, a ring shape, such as a doughnut shape, or any other suitable shape. Preferably, the indicator has a spherical, a cylindrical, or ring shape. Advantageously, the indicator may thereby easily be moved when enclosed in a container holding a perishable product, for instance by tilting or rotating the container. Thereby, the indicator is easily visible to an operator or end-user.

The indicator means is configured to visually indicate an exposure to an undesired condition. The indicator means may comprise a substance 1b. The substance 1b is configured to change color and/or opacity when exposed to an undesired condition. When the indicator means comprises a substance 1b, the shell 1a is preferably piece-wise transparent, transparent or translucent. Upon exposure to the undesired condition, the substance 1b may, for example, change from transparent and colorless to non-transparent and colored. Advantageously, a change in color and/or opacity provides a clear visual indication that the perishable product 8 was exposed to an undesired condition and may be spoiled. Advantageously, a change in color and/or opacity allows an operator or end-user to quickly verify, with one glance, that exposure to an undesired condition has occurred. Consequently, an operator or an end-user may decide to discard the perishable product 8, with minimal waste of time and effort.

The substance 1b may preferably be configured to change to a vibrant color, such as yellow, orange, red, purple, blue or green. The substance 1b may, for instance, e.g. change color from colorless or green, to red, to indicate exposure to an undesired condition. Advantageously, the indicator 1 may thereby function as a ‘traffic light’, where green indicates that the perishable product is safe for use, while red indicates that the perishable product is unsafe for use. The change of color and/or opacity may be irreversible. Alternatively, the change of color and/or opacity may be temporary. In a preferred embodiment, the change of color and/or opacity is irreversible. The substance 1b may comprise a fluid, such as a gel, a liquid, an emulsion, a colloidal dispersion, organic polymers, inorganic polymers or a combination thereof. The substance 1b may, for instance, be configured to undergo a chemical reaction when exposed to an undesired condition, to thereby change color and/or opacity. Alternatively, the substance 1b may comprise a wax, a granular material, a compact or a porous material. For instance, the substance 1b may comprise a solid substance, a frozen solvent, a wax or a polymer configured to melt when exposed to an undesired condition, such as a undesired temperature. Thereby, the substance 1b may change opacity from non-opaque to opaque. Alternatively, the indicator means may comprise several substances including at least one solid substance 1b configured to melt, wherein the melted form promotes reaction of other substances present to give rise to change in color or opacity when exposed to undesired conditions, such as undesired temperatures. The substance 1b, and any further indicator substances, may for instance be configured to undergo a chemical reaction between functional groups, such as, but not limited to, ketones, hydroxyl, phenols, thiols, aldehydes, vinyl azlactones, 2,4-dinitrophenylhydrazine, including acids, bases and pH indicators.

The indicator means may comprise nano sized and/or micron sized magnetic or non-magnetic polymer particles having chemical reactive groups as, but not limited to, e.g. amines, carboxylic, ketones, hydroxy, thiols or aldehydes, attached to the particle surface, capable of further reaction at undesired conditions with other chemical substances as e.g. phenols, 2,4-dinitrophenylhydrazine present within the indicator means, to give a color change. Use of nano sized particles may result in more transparent color systems, whilst use of micron sized particles often results in more opaque systems.

Another embodiment may comprise of reactive initiators attached to the particle surface of the indicator means, where a radical polymerization reaction with monomers present takes place under undesired conditions, e.g. at a given temperature, and where the reaction results in a visual change in color or opacity.

In one embodiment, the indicator means comprises temperature sensitive polymers that either react with other substances present to give a visual color change, change in opacity, a change in a measurable readout or a combination thereof, at undesired conditions. Examples of temperature sensitive polymers are methylcellulose, hydroxypropyl methylcellulose, chitosan, isopropylacrylamide (NIPAAm), polyvinylmethyl ether (PVME), poly(N-vinylisobutyramide) (PNVIBA), polyethyleneoxide-propyleneoxide-b-ethylene oxide (PEO-PPOPEO), Poly(N-isopropylacrylamide) (PNIPAAm), and combination thereof. One may also use pH sensitive polymers as e.g. polyvinyl sulfonic acid, polymethacrylic acid, polydiethylaminoethyl methacrylate, polydimethylaminoethyl methacrylate or a combined pH and a temperature sensitive polymer as poly(N-isopropyl acrylamide-co-butylmethacrylate-co-acrylic acid).

Preferably, the substance 1b changes color and/or opacity when exposed to the undesired condition for a minimal time duration Δt. Preferably, Δt equals the minimal period required for the perishable product 8 to become spoiled, when exposed to the undesired condition. Advantageously, when the perishable product 8 is exposed too briefly to the undesired condition to be spoiled, the indicator 1 may not change color and/or opacity. The perishable product 8 may thereby safely be used by an end-user and not go to waste.

The indicator means may comprise at least one further substance 1c (FIG. 1B). Each substance 1b, 1c may be encapsulated separately by the shell 1a. Alternatively, the substances 1b, 1c may be encapsuled together by the shell 1a. Accordingly, the one or more substances are encapsulated by the shell 1a. Preferably, the substance 1b and at least one further substance 1c are configured to change color and/or opacity when exposed to different undesired conditions, such as different temperatures. Hence, by “different undesired conditions” is meant either different parameters such as temperature and pressure, or the same parameter but at different levels. Different temperatures may, for instance, be the lower end and the upper end of an acceptable temperature range. Each substance may additionally change to the same color and/or opacity, or to a different color and/or opacity. Advantageously, when each substance changes to a different color and/or opacity, it may be easier to detect the cause of the undesired condition retroactively. For example, substance 1b may show that the product has been exposed to too high or too low a temperature, and substance 1c shows that the product has been below the too high or too low temperature for a certain time t which has caused the product to be damaged. In such case the temperature for 1b and 1c may be the same, only that 1c is activated after a longer time than 1b. In another example, substance 1b may show that the product has been exposed to too high or too low a temperature, while 1c shows that the product has been under a too high or too low temperature for a certain time t which has caused the product to be damaged. In that case, the temperature for 1b and 1c is different. In yet a further example, substance 1b measures, for example, too low a temperature and is activated when the product is exposed to such a temperature. Then substance 1c may show that the product has been exposed to elevated temperature but not yet damaged, while a further substance 1c, e.g. a substance called 1d, shows that the product has been damaged because it has been below the elevated temperature for a certain period.

Accordingly, each at least one further substance 1c may change color and/or opacity when exposed to an undesired condition for a different minimal or desired time duration. For instance, one substance 1b may change from colorless or green, to orange, when briefly exposed to and undesired condition. A further substance 1c may then change from colorless to red, when exposed long enough to the undesired condition such that the perishable product 8 may spoil. Alternatively, each at least one further substance 1c may change color and/or opacity when exposed to an undesired condition for a different minimal, defined or desired time duration. For instance, one substance 1b may change from colorless or a predefined color to another color when exposed to the first undesired condition during minimum required exposure time. A further substance 1c may then change from colorless or predefined color to another color when exposed long enough, or after a predefined time, or a desired time, or a required time period, to the second undesired condition. The first undesired condition may be the same, or different from the second undesired condition, e.g. the same temperature or a lower and an upper temperature or a temperature range. Hence, the indicator 1 may comprise a shell 1a, a first indicator 1b, at least one indicator 1c, and a mean for counting or measuring exposure time, or means for counting or measuring exposure time, and or means for activating the at least one indicator 1c. The means for counting or measuring exposure time, and or means for activating the at least one indicator 1c may e.g. be a physical, chemical or electronic timer, reaction or process. The indicator substance 1b may be configured to change color when exposed to a first undesired condition for a minimal time duration Δt1. The indicator 1 may also comprise at least another indicator substance 1c to change color when exposed to a second undesired condition for a minimal time duration Δt2. The indicator substance 1c may change color when exposed to the second undesired condition for a time t1=Δt.

The indicator substance 1c may be further configured to comprise at least another indicator substance 1d (not shown). The indicator 1d may change color when exposed to a second undesired condition for a time t2>Δt. The first and second undesired conditions may be the same or different, e.g. lower and upper temperature or temperature setpoints or ranges. Advantageously, the indicator thereby indicates both that an undesired condition has occurred and whether the undesired condition has persisted long enough for the perishable product 8 to be spoiled. In one embodiment, there is at least one indicator change from exposure of an undesired condition and at least one timed indicator change, e.g at least one change for indicator 1b and/or at least one change for indicator 1c. Advantageously, an operator or end-user may then decide that the perishable product 8 is still useable or may discard the perishable product 8.

Alternatively, and/or additionally, the indicator means may comprise electronic components, as schematically shown in FIGS. 1C, 1D and 1E. The electronic components may comprise a sensor 2 and an energy source 3, preferably placed on an electronic circuit board. The electronic components may further comprise a wireless transmitter 4 and/or a light source 5, such as a light emitting diode (led). Optionally, the electronic components may comprise a storage device (not shown) and/or an RFID chip (not shown) and/or an ID chip (not shown). The sensor 2 is configured to detect a condition, such as temperature, pressure, a particular wavelength of light, such as UV light or IR light, exposure to radioactivity, or a bacterial-, fungal-, or particulate-contamination. Optionally, the indicator means may comprise multiple sensors, each configured to detect a different condition. The energy source 3 is configured to provide energy to the sensor 2, transmitter 4, light source 5 and/or the storage device. The energy source 3 preferably comprises a battery. The transmitter 4 is configured to wirelessly transmit a signal from the sensor 2 and/or from the optional storage device to an external device, detailed below. The transmitter 4 may be any suitable transmitter, such as a radio frequency transmitter. Preferably, the transmitter 4 is a low power transmitter.

The light source 5 is configured to be activated and emit light when the presence of an undesired condition is detected by the sensor 2. Preferably, the light source 5 is irreversibly activated. Once an undesired condition is detected, the light source may therefore continue to emit light. When the electronic components comprise a light source 5, the shell 1a is preferably piece-wise transparent, transparent or translucent. The light source 5 may emit a colored light of a desired color, such as red, blue, green, yellow, orange, purple, or any other color or color combination. The emitted light may comprise a blinking light or a continuous light. The light source 5 may be configured to emit light when exposed to the undesired condition for a minimal time duration Δt. The light source 5 may, for instance, be configured to emit blinking light or light of a first color when exposed to the undesired condition for a time t<Δt. The light source 5 may be further configured to emit continuous light or light of a second color when exposed to the undesired condition for a time t>Δt. Advantageously, the indicator thereby indicates both that an undesired condition has occurred and whether the undesired condition has persisted long enough for the perishable product 8 to be spoiled. An operator or end-user may then decide that the perishable product 8 is still useable or discard the perishable product. Alternatively, if the electronic components comprise multiple sensors, one light source 5 may be provided for each sensor 2.

The indicator means may comprise both electronic components and a substance 1b, as schematically shown in FIG. 1D. Additionally, the indicator means may optionally comprise at least one further substance 1c. Advantageously, the substance 1b may provide a back-up for the electronic components. Should the electronic components fail, the substance 1b may still provide a visual indication for an operator or an end-user. Alternatively, the electronic components and substance 1b may be configured to indicate exposure to different undesired conditions.

Alternatively, the indicator may comprise electronic components and the shell 1a may comprise a non-transparent material, schematically shown in FIG. 1E. The electronic components may comprise a sensor 2, an energy source 3 and a wireless transmitter 4 and optionally a data storage device (not shown), as detailed above. This configuration is advantageous when the perishable product 8 is non-transparent, and a visual indication from the indicator 1 immersed in the perishable product 8 cannot readily be observed.

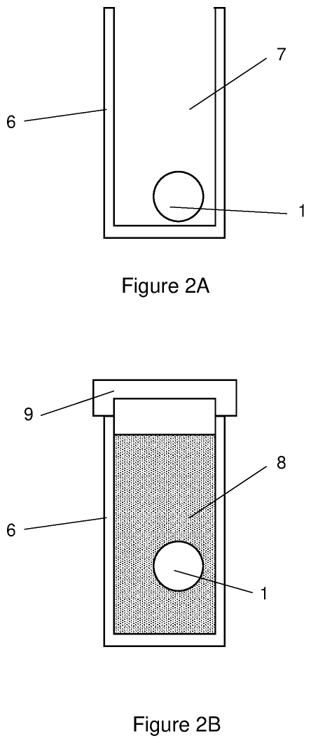

FIG. 2A schematically shows a longitudinal cross-section of a container 6 according to the present disclosure. The container 6 comprises a chamber 7 for holding a perishable product 8. The chamber 7 comprises walls and an opening for filling and emptying. The container 6 may comprise a bag, a vial, a bottle, a box, a cartridge, a syringe, a pen or any other suitable shape. In a preferred embodiment the container is a cartridge, syringe or injector pen. The walls of the chamber 7 may comprise an inorganic polymer as glass, an organic polymer, a metal, or a combination thereof. Preferably, the container is transparent, piecewise transparent or translucent. The container 6 further comprises an indicator 1 according to the disclosure, located in the chamber 7 and freely moveable therein. Hence, at least a wall, or part of a wall of the chamber 7, of the container is transparent or translucent. With freely moveably it is to be understood that the indicator 1 is not fixed to other parts of the container 6. Alternatively, the container 6 may comprise at least one further indicator 1 according to the disclosure.

The indicator 1 may be placed in the chamber 7 before a perishable product 8 is placed therein (shown in FIG. 2A). Alternatively, a perishable product 8 may first be placed in the chamber 7, after which the indicator 1 is placed in the chamber 7. According to yet another alternative, the indicator 1 may be placed in the chamber 7 simultaneously with the perishable product 8. Advantageously, the indicator 1 can therefore be placed in the chamber 7 at any convenient moment during the process of producing and/or filling of the container 6. Improved flexibility in the production and/or filling process is thereby achieved. Furthermore, as the indicator 1 is freely moveable in the chamber 7, the step of adhering or fixing the indicator 1 to the container walls can be omitted. Advantageously, the production process is thereby significantly simplified and less time-consuming. This is especially advantageous for high-volume production processes, for instance during production of medical containers, such as for a medicine, insulin or vaccine vials. The indicator 1 is preferably immersed in the perishable product 8. Advantageously, the indicator 1 thereby provides a more accurate indication of the undesired condition. The density of the indicator 1 may be configured to be greater than the density of the perishable product 8. The indicator 1 may therefore sink to the bottom of the chamber 7, when the container 6 is held in an upright position. Alternatively, the density of the indicator 1 may be equal to or less than the density of the perishable product 8, so that the indicator 1 may remain suspended in the perishable product 8 or may float at the surface of the perishable product 8.

The container 6 may further comprise a lid 9, schematically shown in the longitudinal cross-section of FIG. 2B, for closing and sealing of the chamber 7. The lid 9 may comprise a glass, a polymer, a metal, or a combination thereof. After placing a perishable product 8 and an indicator 1 in the chamber, the chamber 7 is closed by the lid 9, in a fluid tight manner. The lid 9 may be screwed, clamped, adhered and/or welded onto or into the chamber 7. Alternatively, the walls of the chamber 7 may be sealed directly together or different parts are mounted together and sealed with e.g. sealing means such as o-rings, sealing lips, or similar methods to close the chamber 7, for instance when the container 6 comprises cylinder and piston, syringe, pen, bag or box.

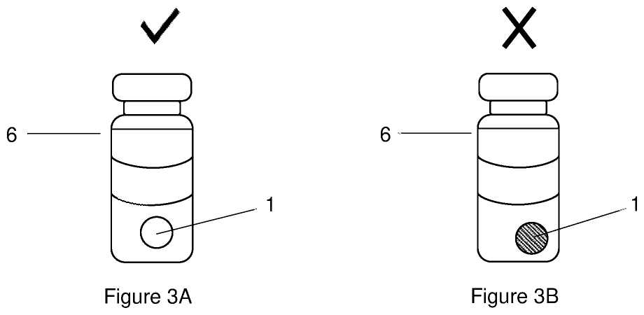

The indicator 1 according to the present disclosure may be used during the transport and/or storage of a perishable product 8. The indicator 1 is configured to be immersed and freely-moveable within the perishable product 8. Preferably, the indicator 1 comprises a spherical shape, a cylindrical shape or o-ring shape and a fluid-tight shell 1a. Furthermore, the container 6 according to the present disclosure may be used for the transport and/or storage of a perishable product 8. The perishable product may comprise a medical fluid, such as a vaccine, an insulin, a biological sample, blood, a medicine, or an intravenous fluid. In one embodiment, the product is a medical fluid. In one embodiment, the medical fluid is insulin. Alternatively, the perishable product 8 may comprise a food product, such as a dairy product, an egg-based product or a beverage. Use of the container 6 is shown in FIGS. 3A and 3B, e.g. for a vaccine. In FIG. 3A the indicator 1 has not changed color and the product, such as vaccine, medicine or insulin can safely be used. In FIG. 3B the indicator 1 has irreversibly changed color; the product, such as vaccine, medicine or insulin is spoiled and should preferably be discarded.

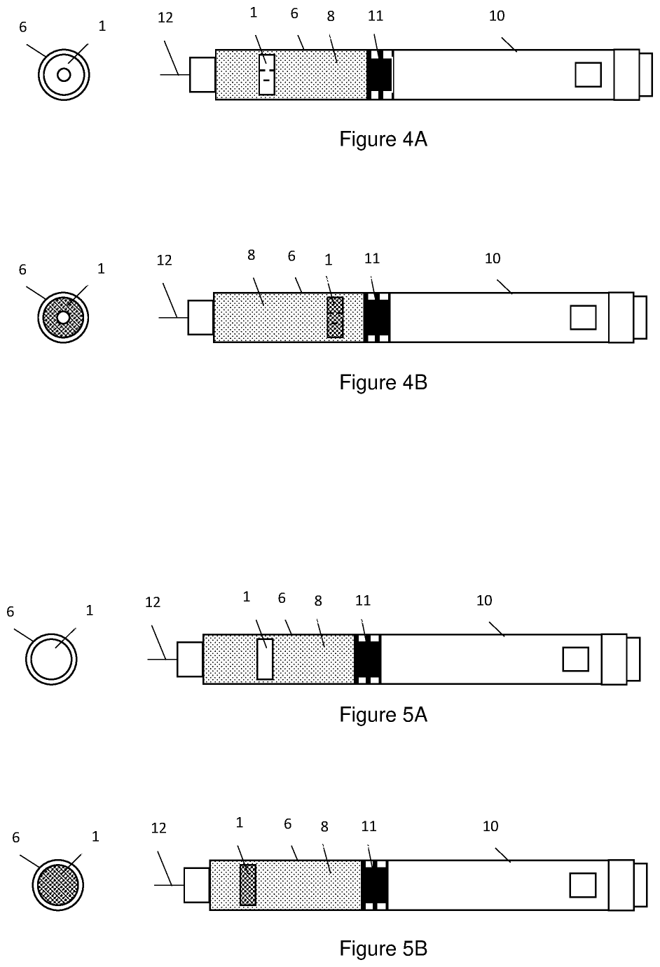

Alternative containers and indicators according to the invention, for use for a medical product, are shown in FIGS. 4 and 5, in sideview and corresponding cross-sectional view. A container 6 in the form of a syringe cartridge (pen) 10 is provided in FIGS. 4 and 5, wherein in FIGS. 4A and 4B the indicator means 1 has a ring shape, i.e. having an annular shape. The cartridge 10 includes a piston 11 and a needle 12. The indicator 1 is positioned in the cartridge being freely movably in the medical product 8. In one embodiment, the product is insulin. Hence, the medical product can be squeezed out through the opening of the annularly shaped indicator. The indicator can move freely in the chamber 7 and within the product. In FIG. 4A the indicator has not changed color, while in FIG. 4B the indicator has changed color as a result of being exposed to an undesired condition. In FIGS. 5A and 5B the indicator means 1 has a cylindrical shape. The indicator is positioned in the cartridge being freely movably in the medical product. The medical fluid, e.g. insulin, can be forced out around the indicator cylinder as it has a smaller outer diameter than the inner diameter of the container. There is hence a gap between the indicator cylinder and the product reservoir. In FIG. 5A the indicator has not changed color, while in FIG. 5B the indicator has changed color as a result of being exposed to undesired conditions. The invention is not limited to the illustrations shown in FIGS. 1-5 as the figures are intended as examples only and used for illustration purposes.

A system for a perishable product 8 may comprise an indicator 1 according to the disclosure and an external device (not shown). The indicator 1 comprises a transmitter 4, as detailed above. Optionally, the indicator 1 comprises an RFID chip. The external device is configured to receive the signal from the transmitter 4 and/or to read the RFID chip and to convert the signal to at least a visual indication. Thereto, the external device may comprise a screen, at least one light source, or combinations thereof. The light source may be at least one led. Preferably, the screen or at least one light source is irreversibly activated. Once an undesired condition is detected, the screen or light source may therefore continue to provide the visual indication. The visual indication preferably comprises a vibrant color, such as yellow, orange, red, purple, green or blue, or other colors, or any color combination thereafter. Optionally, the visual indication may be a blinking indication or a continuous indication. Advantageously, the external device thereby indicates that the indicator 1 was exposed to an undesired condition. The signal from the transmitter 4 may be encoded and the external device may be provided with a decoding key. Advantageously, the communication between the indicator and the external device can thereby be kept exclusive, such that only an operator or end-user with an approved external device can read the indicator signal. Optionally, a detected condition stored on the storage device may be transmitted by the transmitter 4 to the external device. Advantageously, a condition history of the perishable product 8 can thereby be provided to an operator or an end-user. The moment of occurrence of an undesired condition may thereby precisely established.

In one embodiment the ID is used as an RFID without an antenna. The unit thus becomes smaller. To read the content of the ID chip, the indicator must be in close contact with the reader. As an example, the indicator can have superparamagnetic properties, i.e. when decided the indicator is pulled down on a magnet. The indicator is otherwise freely movable in the perishable product. Data is read and made visible as desired.

A method for producing the indicator 1 may comprise providing indicator means and moulding the shell 1a to encapsulate the indicator means. The indicator means may comprise a substance 1b, and optionally at least one further substance 1c. Moulding the shell 1a may comprise injection moulding, extrusion moulding, blow moulding or any other suitable moulding technique. After moulding, the shell is hardened. Inserting the substance 1b and the optional at least one further substance 1c may, for instance, comprise injecting the substance 1b into the shell 1a before hardening of the shell 1a. The method may include a step of adjusting the density and buoyancy of the indicator means to the product which it is to be used in. The indicator 1 may be further configured to comprise a mean for activating the indicator at a desired time or condition—prior to the insertion into the perishable product or when the monitoring needs to start.

Alternatively or additionally, providing the indicator 1 may comprise providing electronic components, positioning the electronic components in a mould and moulding the shell 1a to enclose the electronic components. Preferably, the moulding comprises injection moulding. The electronic components may be enclosed in a protective film before positioning in the mould. Advantageously, the electronic components are thereby protected during the moulding process.

The method may further comprise providing a container 6 comprising a chamber 7, optionally, sterilizing at least one indicator 1 and/or the container 6, and inserting the at least one indicator 1 in the chamber 7. The container 6 may be filled with a perishable product 8 before the step of inserting the at least one indicator 1, during the step of inserting the at least one indicator 1, or after the step of inserting the at least one indicator 1. The container 6 may next be closed and sealed, for instance by closing and sealing the container with a lid 9 or by closing and sealing the container walls together directly, without the use of a lid.

### EXAMPLES

### Example 1: Preparation of Indicator Means for Color Change at 0° C. Using Acid/Base Systems

Reagent 1: NaOH (0.1 M) and Reagent 2: Citric acid (0.033 M)+Phenolphthalein, were chilled to 0° C., and 1 ml of reagent 1 was introduced into the vial. The vial was chilled to −80° C., and the solution allowed to harden (30 min). 1-2 layers of water (each layer 1 ml) were frozen on top of reagent 1 in the same way. Finally, reagent 2 was introduced and frozen. The indicators were then stored at either −20 or −80° C. until needed. On heating, the indicator turned red in a temperature interval −0.5° C.-0° C. Re-freezing of indicator showed no color change.

### Example 2: Preparation of Indicator Means for Color Change at −15° C. Using Acid/Base Systems

Reagent 1: NaOH (0.1 M) and Reagent 2: Citric acid (0.033 M)+Phenolphthalein, were chilled to 0° C., and 1 ml of reagent 1 was introduced into the vial. The vial was chilled to −80° C., and the solution allowed to harden (30 min). 1-2 layers of a solution DI water:ethanol=40:60 (each layer 1 ml) were frozen on top of reagent 1 in the same way. Finally, reagent 2 was introduced and frozen. The indicators were then stored at either or −80° C. until needed. On heating, the indicator turned red in a temperature interval −15° C.-−14° C. Re-freezing of indicator showed no color change.

### Example 3: Preparation of Indicator Means for Color Change at Interval 2-4° C. Using Schiff Reagent Systems

Reagent 1: Schiff reagent and Reagent 2: Glutaraldehyde aq. solution (50% vol.), were chilled to 0° C., and 1 ml of reagent 1 was introduced into the vial. The vial was chilled to −80° C., and the solution allowed to harden (30 min). 1-2 layers of DI water (each 1 ml) were frozen on top of reagent 1 in the same way. Finally, reagent 2 was introduced and frozen. The indicators were then stored at either −20 or −80° C. until needed. On heating, the indicator turned purple at a temperature interval 2° C.-4° C. Re-freezing of indicator showed no color change.

### LIST OF REFERENCES

1: indicator
1a: shell
1b: substance
1c: further substance
2: sensor
3: energy source
4: wireless transmitter
5: light source
6: container
7: chamber
8: perishable product
9: lid
10: syringe or pen
11: piston or emptying mechanism
12: needle

1-20. (canceled)

21. An indicator for a perishable product, the indicator comprising:
indicator means, configured to irreversibly, visually indicate an exposure to an undesired condition related to perishability of the product; and
a fluid-tight and transparent, piecewise transparent, or translucent shell encapsulating the indicator means;
wherein the indicator is configured to be immersed and freely-moveable in the perishable product.

22. The indicator of claim 21, wherein the indicator means comprises a substance, the substance being configured to change color and/or opacity when exposed to a first undesired condition.

23. The indicator of claim 22, wherein the indicator means comprises a further substance, the further substance being configured to change color and/or opacity when exposed to a further undesired condition, the further undesired condition being different from the first undesired condition, or the substance being configured to change color and/or opacity when exposed to an undesired condition for a minimal, defined or desired time duration different from that of the first substance.

24. The indicator of claim 23, wherein the substance and the further substance individually comprise any one of a granular material, a porous material, a fluid or a combination thereof.

25. The indicator of claim 21, wherein the undesired conditions are a temperature or a pressure outside an acceptable range, or a light with an undesirable wavelength.

26. The indicator of claim 21, wherein the indicator indicates both that an undesired condition has occurred and whether the undesired condition has persisted long enough for the perishable product to be spoiled.

27. The indicator of claim 21, wherein the indicator means comprises a sensor, an energy source and a light source; and wherein the light source is configured to provide a visual indication when the sensor detects an undesired condition.

28. The indicator of claim 21, wherein the indicator means comprises a sensor, an energy source and a wireless transmitter; and wherein the wireless transmitter is configured to transmit a signal when the sensor detects an undesired condition.

29. The indicator of claim 21, wherein the shell comprises a glass or a polymer.

30. The indicator of claim 21, wherein the indicator comprises a spherical shape, a cylindrical shape, a ring shape, a conical shape or a cuboid shape.

31. A container for a perishable product, the container comprising:
a chamber, configured to hold the perishable product; and
at least one indicator according to claim 21;
wherein the at least one indicator is located within the chamber being freely moveable therein.

32. The container of claim 31, wherein the chamber further comprises a perishable product, the perishable product comprising:
a medical fluid, such as a vaccine, a biological sample, blood, a medicine, or an intravenous fluid; or
a food product, such as a dairy product, an egg-based product or a beverage.

33. The container of claim 31, wherein the chamber comprises a perishable product, the perishable product being insulin.

34. The container of claim 31, wherein the container comprises either of a bag, a vial, a bottle, a box, a cartridge, a syringe, or an injector pen, and is more preferably a cartridge, syringe or injector pen.

35. The container of claim 31, wherein the chamber is closed and sealed, preferably by a lid.

36. Use of an indicator according to claim 21 or of a container for transport and/or storage of a perishable product, the container comprising (i) a chamber configured to hold the perishable product and (ii) the indicator, wherein the indicator is located within the chamber being freely moveable therein, and the perishable product comprising:
a medical fluid, such as a vaccine or insulin, a biological sample, blood, a medicine, or an intravenous fluid; or
a food product, such as a dairy product, an egg-based product or a beverage.

37. A method of producing an indicator according to claim 21, the method comprising:
providing indicator means, configured to indicate an exposure to an undesired condition; and
molding a shell such that the shell encapsulates the indicator means.

38. The method of claim 37, further comprising:
providing a container comprising a chamber;
optionally, sterilizing the at least one indicator and/or the container; and
inserting the at least one indicator into the chamber so that the at least one indicator is freely moveable therein.

39. The method of claim 38, further comprising:
filling the container with a perishable product before the step of inserting the at least one indicator, during the step of inserting the at least one indicator, or after the step of inserting the at least one indicator; and
closing and sealing the container.

40. The method of claim 39, wherein the container is a cartridge, syringe or injector pen, and the perishable product is insulin.
